(19) United States
(12) Patent Application Publication
(10) Pub. No.: US 2022/0409682 A1
(43) Pub. Date: Dec. 29, 2022

(54) Aqueous liquid extract of Spirulina for the prevention and/or treatment of chemically induced peripheral neuropathy and their symptoms, composition and use thereof

(71) Applicant: ALGOSOURCE, Saint-Nazaire (FR)

(72) Inventor: Olivier Lépine, Saint-Nazaire (FR)

(21) Appl. No.: 17/777,861

(22) PCT Filed: Nov. 19, 2020
(86) PCT No.: PCT/EP2020/082661

§ 371 Date: May 18, 2022

(30) Foreign Application Priority Data
Nov. 19, 2019 (FR) FR1912891

(51) Int. Cl. A61K 35/748; A61K 38/16; A61P 25/02

(52) U.S. Cl. CPC A61K 35/748; A61K 38/164; A61P 25/02; A61K 2236/331

(57) ABSTRACT

A composition for use in the treatment and/or prevention of adverse side effects induced by an anti-cancer compound, said composition comprising an aqueous liquid extract, the aqueous liquid extract comprising Phycocyanin. The present invention also relates to the use of this aqueous liquid extract comprising Phycocyanin and optionally Spirulina polysaccharides, for the treatment and/or prevention of adverse side effects induced by an anti-cancer compound.

Indeed, Phycocyanin, thanks to its antioxidant effects, can reduce the undesirable side effects induced by an anti-cancer compound.

### TECHNICAL FIELD OF THE INVENTION

The field of the invention is that of the treatment and/or prevention of undesirable side effects induced by an anti-cancer therapeutic treatment, and in particular a chemotherapeutic treatment.

More particularly, the invention relates to the treatment and/or prevention of undesirable side effects induced by the administration of an anti-cancer compound, and in particular the prevention and/or treatment of peripheral neuropathy(ies) induced by chemotherapies, or chemotherapy-induced neuropathy(ies).

More specifically, the invention relates to the use of an aqueous liquid extract obtained from cyanobacteria, or Spirulina, containing Phycocyanin and optionally Spirulina polysaccharides, for the prevention and/or treatment of chemotherapy-induced peripheral neuropathy(ies); a composition comprising said aqueous liquid extract for use in the prevention and/or treatment of undesirable side effects induced by anti-cancer therapeutic treatment, and in particular chemotherapy-induced peripheral neuropathy.

### PRIOR ART

Chemotherapy is currently the most commonly used procedure in cancer treatment. It can be used alone or in combination with other therapeutic approaches (surgery, radiotherapy, targeted therapy, etc.).

Chemotherapy is a treatment involving the administration of drugs that act on cancer cells, either by destroying them or by preventing them from multiplying. However, these drugs also act on other cells in the body that are growing rapidly, and this explains the unwanted side effects of chemotherapy.

Indeed, the side effects are linked to the action of anti-cancer drugs on cells that multiply rapidly, i.e. cancer cells but also those of the bone marrow, hair, skin, etc. The side effects of chemotherapy are frequent and vary in intensity depending on the drugs used, the dosage, the method of administration and the individual.

“Side effect” means an adverse effect of a drug or care that induces or potentially induces an adverse effect by the treatment (e.g. discomfort, allergy, serious complications, including death). This side effect can be immediate or delayed.

The most common side effects are changes in the blood count (decrease in certain white blood cells, red blood cells and platelets), digestive problems (nausea, vomiting, diarrhoea, constipation), hair loss, fatigue, numbness or tingling sensations. Other side effects are possible such as kidney, heart or liver problems. For example, it is known that platinum salts (cisplatin, carboplatin, and oxaliplatin) and taxanes (paclitaxel and docetaxel) are neurotoxic and cause the development of neuropathy in patients. Anthracyclines (epirubicin, pirarubicin, idarubicin, zorubicin, aclarubicin, doxorubicin) are known to promote lipid peroxidation and to be cardiotoxic.

Chemotherapy-induced neuropathy is among the most severe side effects. Chemically induced neuropathy means neuropathy caused by administration of or exposure to a neurotoxic compound, where the compound may be:
either a medicinal compound, in particular an anti-cancer compound presenting a neurotoxicity;
or a non-medicinal compound such as alcohol, organophosphates, heavy metals (Pb, As, Hg, TI), fungal or bacterial toxins, certain solvents like toluene, etc.

This type of neuropathy is characterised by a mainly sensory attack, persistence of paraesthesiae and sensations of numbness between courses of chemotherapy, as well as spontaneous pain. In the most severe cases, patients develop sensory ataxia accompanied by a loss of superficial and deep sensitivity affecting the smallest gestures of daily life.

Neuropathic pain, like allodynia, hyperesthesia, dysesthesia and ataxia, is a symptom of neuropathy, defined as a pain secondary to an injury or condition affecting the somatosensory system. It is secondary to peripheral lesions (afferent nerve fibres) or central lesions (nerve centres involved in the transmission and/or regulation of the pain message, for example in the thalamus).

The etiologies of neuropathic pain are diverse. They can be caused by a physical injury such as in an accident, a surgical procedure, an amputation (phantom limb). They can also be chemically induced as is the case following alcohol abuse, metal poisoning (arsenic or thallium for example), exposure to an environmental chemical agent (organophosphate for example), a drug, a fungal or bacterial toxin.

They may also result from an acquired or inherited medical condition (e.g. amyloidosis, Guillain-Barré syndrome, HIV infection, bacterial infection, shingles, herpes, diabetes, autoimmune disease and/or Fabry disease).

With regard to neuropathy (particularly neuropathic pain and associated sensitivity disorders) induced by chemotherapy such as that with platinum salts, it would seem that it is directly related to the duration of the treatment and the dose administered.

In order to limit these undesirable side effects, dosage adjustments are generally made, i.e. a reduction in the doses administered, a spacing out of the treatments, or even the interruption of the treatments. This dosage adjustment compromises the patient's chances of remission and survival. This is the case, for example, for limiting or even eliminating the symptoms of neuropathy induced by platinum salt treatments.

Phycocyanin, or C-Phycocyanin, is a protein of the phycobiliprotein family characterised by its antioxidant activity (“C-Phycocyanin prevents cisplatin-induced nephrotoxicity through inhibition of oxidative stress.” Fernandez-Rojas B. et al, Food Functio 5:480-490 (2014)) and by an intense blue colour (due to a characteristic absorption peak at 620 nm). Phycocyanin is one of the main pigments of a prokaryotic micro-alga, or cyanobacteria, commonly called Spirulina, used as a dietary supplement because of its high protein content.

Indeed, Phycocyanin is used in the food industry, cosmetics, biotechnology and medical practice. Some of its nutraceutical properties are antioxidant, anti-inflammatory, anti-neoplastic and anti-diabetic. Indeed, its antioxidant activity is involved in the protective effect against liver, neuronal and renal lesions. In particular, Phycocyanin has been shown to have several nutraceutical properties such as renoprotective (against oxalate and cisplatin) (“Salubrious effect of C-phycocyanin against oxalate-mediated renal cell injury.” Farooq S et al, Clin Chim Acta 348:199-205 (2004); “C-Phycocyanin attenuates cisplatin-induced nephrotoxicity in mice.” Lim B J, et al, Ren Fail 34:892-900 (2012)), anti-inflammatory (“Further studies on anti-inflammatory activity of phycocyanin in some animal models of inflammation.” Romay C et al, Inflamm Res 47:334-338 (1998)) and hepatoprotective (against carbon tetrachloride) (“Protective effect of C-Phycocyanin against carbon tetrachloride-induced hepatocyte damage in vitro and in vivo” Ou Y, et al, Chem Biol Interact 185:94-100, (2010)).

It has also been shown that Phycocyanin plays a protective role against certain side effects of anti-cancer compounds such as cisplatin. In particular a protective role of Phycocyanin against cisplatin-induced renal mitochondrial dysfunction has been demonstrated, when administered at a dose of 22-30 mg/kg body weight (“C-Phycocyanin prevents cisplatin-induced mitochondrial dysfunction and oxidative stress”, Fernandez-Rojas B. et al, Mol Cell Biochem (2015)).

Finally, Phycocyanin, when administered at a dose of 5-30 mg/kg body weight, has been shown to play a role in preventing oxidative stress and decreasing the activity of antioxidant enzymes in cisplatin-induced nephrotoxicity.

Hence, this data suggests that human consumption of Phycocyanin may be useful for the prevention and/or treatment of renal diseases associated with oxidative stress induced by chemotherapeutic treatment involving cisplatin. (“C-Phycocyanin prevents cisplatin-induced nephrotoxicity through inhibition of oxidative stress” Berenice Fernandez-Rojas et al, Food Funct, 2014).

Until now, treatments to reduce chemotherapy-related side effects have been aimed at individual side effects (e.g. nausea, vomiting, hair loss, etc.) and have not necessarily been able to act further upstream to treat their cause that could be common (e.g. induction of oxidative stress leading to the cell death of healthy cells) in an effective and targeted manner.

Phycocyanin, thanks to its antioxidant activity, can reduce some side effects linked to anti-cancer compounds such as cisplatin. However, a protective effect of Phycocyanin is only observed at very high dosages between 350-900 mg for an adult of about 70 kg, which is constraining in terms of cost and production on an industrial scale, but also has consequences on the treatment of the patient (for example high dosage).

### PURPOSES OF THE INVENTION

One of the objectives of the invention is to overcome these drawbacks of the prior art and to propose a therapeutic alternative to simple dosage adaptation.

More specifically, the purpose of the invention, in at least one embodiment, is to provide a composition for preventing and/or eliminating, or at least further reducing, undesirable side effects induced by an anti-cancer compound.

In particular, the purpose of the invention, in at least one embodiment, is to provide a composition for preventing and/or eliminating, or at least reducing, peripheral neuropathy induced by the administration of an anti-cancer compound, and in particular an anti-cancer compound selected from platinum salts.

Another purpose of the invention, in at least one embodiment, is to provide a composition for preventing and/or eliminating, or at least further reducing, the undesirable side effects induced by an anti-cancer compound with a lower Phycocyanin dosage than the prior art.

### DISCLOSURE OF THE INVENTION

These objectives, and others which will appear hereafter, are achieved by using a composition for use in the treatment and/or prevention of chemotherapy-induced peripheral neuropathy and its symptoms induced by an anti-cancer compound, said composition comprising an aqueous liquid extract, said aqueous liquid extract comprising Phycocyanin.

Indeed, Phycocyanin, thanks to its antioxidant effects, can reduce the undesirable side effects induced by an anti-cancer compound.

According to a first particular characteristic, the aqueous extract further comprises Spirulina polysaccharides.

Advantageously, Spirulina polysaccharides potentiate the effect of Phycocyanin making the aqueous liquid extract according to the invention particularly effective in the treatment and/or prevention of side effects induced by an anti-cancer compound and in particular chemotherapy induced peripheral neuropathy and their symptoms.

Indeed, the inventors have unexpectedly brought to light the fact that such a composition comprising an aqueous liquid extract of cyanobacteria, also known under the name Spirulysat® and marketed by the company AlgoSource, when taken as an adjunct to an anti-cancer therapeutic treatment, such as, in particular, a chemotherapy treatment, makes it possible to reduce the undesirable side effects of this chemotherapy treatment even more effectively.

Hereafter, the term Spirulysat® is used to designate the aqueous liquid extract according to the invention, i.e. comprising Phycocyanin and optionally Spirulina polysaccharides.

One positive consequence of this significant reduction in undesirable side effects related to cancer treatments is the possibility of subjecting patients undergoing cancer treatment, such as a chemotherapy, whether or not combined with radiotherapy, to all the cycles required for treatment.

In addition, patients whose state of health no longer allows them to receive heavy treatments (chemotherapy combined or not with radiotherapy) have seen their condition stabilise thanks to Spirulysat®. Thus, the inventors have highlighted that the administration of Spirulysat®, without constituting a curative treatment, may offer patients a significant additional chance of survival.

Moreover, Spirulysat® is very well accepted by all patients even in case of severe digestive disorders (nausea, vomiting, abdominal pain, diarrhoea, etc.).

The inventors put forward that the role of Spirulysat® in the reduction of undesirable side effects related to anti-cancer therapeutic treatments, such as chemotherapy, would be due in particular to its powerful antioxidant functionalities linked to the presence of Phycocyanin. Indeed, the antioxidant properties of Spirulysat® limit oxidative stress, which helps to protect organs during chemotherapy.

The antioxidant functionalities of Spirulysat were notably demonstrated through the “Spirox” study conducted by the companies AlgoSource and Biofortis Merieux NutriSciences (“Study to Assess Antioxidant Efficacy of Spirulina on oxLDL and Lipids Metabolism on Subjects With Metabolic Syndrome (SPIROX)”, 2016). This clinical study was conducted in France on 40 people to demonstrate the antioxidant activity of Spirulysat®. In particular, this study showed the effect of Spirulysat® on oxidative stress and lipid metabolism in subjects with metabolic syndrome. This study was conducted double-blind against placebo, with a conclusive result on the urinary isoprostanes marker (lipid oxidation marker). This study demonstrates the antioxidant activity of Spirulysat® for two ampoules per day (for 12 weeks): 20 mg of Phycocyanin per day and 3 mg of Spirulina polysaccharides per day.

Indeed, taking Spirulysat® in addition to an anti-cancer treatment, such as chemotherapy, stimulates the immune system, protects organs such as kidneys, liver, nerves or neurons, which has the effect of reducing the appearance of undesirable side effects. In particular, the inventors have demonstrated a role for Spirulysat® in reducing chemically-induced peripheral neuropathy and its symptoms.

These undesirable side effects belong to the group of immune system disorders, kidney disorders, liver disorders, heart disorders and chemically-induced peripheral neuropathy and their symptoms, or their combination.

In one example, the action of Spirulysat® on the liver has been demonstrated in particular by a study conducted at the west France human nutrition research centre and published in the journal Nutrients (Marni Coué et al, “Spirulina Liquid Extract Protects against Fibrosis Related to Non-Alcoholic Steatohepatitis and increases Ursodeoxycholic Acid”, Nutrients 2018). The disorders on the liver due to oxidative stress of chemical nature or by a hypercaloric diet have been prevented by Spirulysat®.

In particular, according to a characteristic of the invention, the undesirable side effects are selected from chemotherapy-induced peripheral neuropathy and their symptoms.

According to another characteristic, the anti-cancer compound belongs to the group comprising a platinum salt, a taxane, an anthracycline.

Advantageously, Spirulysat® allows to treat and/or prevent undesirable side effects related to chemotherapies. In particular, benefits of the composition according to the invention have been observed on kidney and brain function during chemotherapies involving platinum salts, such as cisplatin, oxaliplatin and carboplatin.

Similarly, a protective effect has been observed on the brain, mainly during oncological treatment with taxanes, such as paclitaxel and docetaxel.

A protective effect on the heart has also been observed during anthracycline-based oncology treatment, such as epirubicin.

More particularly, the anti-cancer compound is selected from the platinum salts; preferably from the group comprising cisplatin, carboplatin, oxaliplatin and their mixture.

Advantageously, the composition according to the invention makes it possible to reduce the undesirable side effects of platinum salt-based chemotherapies, and more specifically, chemotherapies involving oxaliplatin.

According to another characteristic of the invention, the liquid aqueous extract comprises between 20 and 150 mg, preferably between 50 and 120 mg, very preferably 100 mg of Phycocyanin.

Preferably, the liquid aqueous extract further comprises between 5 and 25 mg, very preferably 20 mg of Spirulina polysaccharides.

According to another characteristic, the composition comprises only said liquid aqueous extract and is preferably administered in liquid form orally.

Advantageously, the composition according to the invention is administered orally, which allows it to be well tolerated by the patient and to be simple to administer. Moreover, this composition is taken as a food supplement that does not require medical assistance.

Preferably, the composition comprising only said liquid aqueous extract is administered at a dose between 20 mg/day and 150 mg/day, preferably between 50 mg/day and 150 mg/day, very preferably at 100 mg/day.

Thus, the composition according to the invention makes it possible to obtain effects on the reduction of side effects induced by anti-cancer compounds, and in particular peripheral neuropathies induced by platinum salts, with doses of Phycocyanin at least 6 to 18 times lower than those described in the prior art.

Advantageously, several levels of Spirulysat concentrations were tested, and an effect/dose relationship was observed.

In particular, the inventors have demonstrated a very significant action of Spirulysat® concentrated at a dose 2 to 5 times higher than the values of 20 mg/day and 50 mg/day, in the reduction of numerous side effects of chemotherapeutic treatments.

The invention also relates to the use of an aqueous liquid extract, containing Phycocyanin and optionally polysaccharides, obtained from cyanobacteria for the treatment and/or prevention of chemotherapy-induced peripheral neuropathy and their symptoms induced by an anti-cancer compound.

Preferably, the anti-cancer compound belongs to the group comprising a platinum salt, a taxane, an anthracycline, in particular the anti-cancer compound is selected from the platinum salts; preferably from the group comprising cisplatin, carboplatin, oxaliplatin and their mixture.

### DETAILED DESCRIPTION OF THE EMBODIMENTS

The general principle of the invention is based on the administration of a composition comprising an aqueous liquid extract obtained from cyanobacteria, said aqueous extract comprising Phycocyanin and optionally Spirulina polysaccharides, for use in the treatment and/or prevention of undesirable side effects induced by an anti-cancer compound, in particular to prevent and/or treat chemotherapy-induced peripheral neuropathy and their associated symptoms, such as neuropathic pain.

The invention relates more particularly to a composition comprising only the aqueous extract for use in the treatment and/or prevention of peripheral neuropathy induced by platinum salt therapy such as oxaliplatin.

In particular, this aqueous liquid extract of cyanobacteria, or Spirulina, comprises Phycocyanin and very preferably Spirulina polysaccharides, and is marketed by the Company AlgoSource under the name Spirulysat®. Spirulysat® is a liquid aqueous extract, the extraction process of which is described in the patent application FR3064269. This particular extraction process allows the extraction of Phycocyanin without denaturing it, i.e. by preserving its spatial structure. Thus Spirulysat® is particularly rich in Phycocyanin of better quality, which gives it an increased effectiveness.

This liquid aqueous extract is obtained from cyanobacteria, or micro-algae. In particular, Spirulysat® is obtained from cyanobacteria selected from Arthrospira platensis spirulina, Aphanizomenon flos-aquae and Phormidium molle. Cyanobacteria are prokaryotic microalgae also called “blue-green algae”. The term “Spirulina” can refer to several different species of filamentous cyanobacteria, such as species from two distinct genera: Arthrospira and Spirulina. Hereafter, the term “Spirulina” is used to describe the cyanobacteria used to obtain Spirulysat®. Spirulina is known to be very rich in antioxidant molecules such as Phycocyanin, a phycobiliprotein of an intense blue.

It is understood that “polysaccharides” (sometimes called glycans, polyosides, polyholosides or complex carbohydrates) are polymers of the carbohydrate family consisting of several simple sugars linked together by osidic bonds.

In an embodiment according to the invention, the liquid aqueous extract comprises between 20 and 150 mg, preferably between 50 and 120 mg, very preferably 100 mg of Phycocyanin. Preferably, the liquid aqueous extract further comprises between 5 and 25 mg, very preferably 20 mg of Spirulina polysaccharides.

Spirulysat® is preferably administered in liquid form orally. In particular, the composition is administered at a dose between 20 mg/day and 150 mg/day, preferably between 50 mg/day and 150 mg/day, very preferably at 100 mg/day.

In particular, the invention comprises, in one of its particularly interesting aspects, the administration of the composition comprising Spirulysat® upstream and/or in parallel with the administration of an anti-cancer compound, in particular an anti-cancer compound selected from platinum salts, taxanes, anthracyclines or their mixture.

Platinum salts such as oxaliplatin, carboplatin and cisplatin are currently used to treat certain forms of cancer. They all present a more or less important neurotoxicity depending on the molecule, oxaliplatin being the most neurotoxic of the platinum salts. This neurotoxicity is translated by the appearance of signs encountered in neuropathic pain such as burning, tingling, numbness, allodynia, paraesthesia, which persist long after the treatment is stopped. This neurotoxicity occurs acutely following each administration of oxaliplatin to the patient, and becomes chronic as the patient receives doses of platinum salts (cisplatin, carboplatin and oxaliplatin).

In a preferred embodiment, Spirulysat® is administered prior to the administration of the anti-cancer compound, in particular prior to the administration of platinum salts, such as oxaliplatin. This option potentiates the effects of Spirulysat® by giving it time to act and avoiding the development of peripheral neuropathy and associated symptoms. Prophylactic administration of Spirulysat®, prior to the administration of at least one anti-cancer compound, notably a platinum salt such as oxaliplatin, helps to avoid the development of peripheral neuropathy and associated symptoms, such as neuropathic pain.

Advantageously, Spirulysat® is not administered on the day of administration of the anti-cancer compound, in particular a platinum salt, in order to allow the anti- cancer treatment to act, nor on the day following this administration.

Administration of Spirulysat® is resumed two to three days after administration of the anti-cancer compound, including a platinum salt, and every day until the next administration of the anti-cancer compound, including a platinum salt.

The inventors observed that the administration of a dose of Spirulysat® before the start of anti-cancer treatment including the injection of platinum salts, and then daily between injections of platinum salts, made it possible to reduce the appearance of peripheral neuropathy caused by these platinum salts, and prevent the development of neuropathy and thus of chronic pain.

In an example of administration of Spirulysat®, it is administered in the form of an ampoule comprising 10 ml of the aqueous liquid extract, i.e. approximately 10 mg of Phycocyanin and 1.5 mg of Spirulina polysaccharides. The invention is not limited to this mode of administration, nor to the composition and/or volume of the ampoule.

In another example of administration of Spirulysat®, it can be administered as a 5 ml ampoule of the aqueous liquid extract.

In other examples of administration, Spirulysat® can be administered in a glass bottle, as a capsule containing the aqueous liquid extract, or as a drink containing the aqueous liquid extract.

In fact, with an intake of 2 ampoules/day, i.e. about 20 mg/day of Phycocyanin and 3 mg/day of Spirulina polysaccharide, effects have been observed, but in a limited way in certain patients.

In humans, during chemotherapeutic treatment, especially treatment with platinum salts and more particularly oxaliplatin, a dose of 5 to 6 ampoules per day, i.e. 50 mg-60 mg/day of Phycocyanin and 7.5 mg/day-9 mg/day of Spirulina polysaccharides, gives much better results than a dose of 2 ampoules per day, i.e. 20 mg/day of Phycocyanin and 3 mg/day of Spirulina polysaccharides.

On pets such as cats, dogs, hamsters, exotic pets, a dose of 0.1 ml of Spirulysat® per kg of body weight has been identified as active. This corresponds to the same orders of magnitude: 7 ml for 70 kg. Doses equivalent to those administered to humans and therefore higher have more effects. Thus, in animals have also observed a protective effect of Spirulysat® particularly on the liver, kidneys, heart or nervous system.

Thus, Spirulysat® gives effects from 32 mg of Phycocyanin, which is 10 times less than what is described in the prior art, i.e. 350 mg-900 mg of Phycocyanin, which makes it very affordable from an industrial point of view, but also for the patient.

Advantageously, a dose of 10 ampoules per day, i.e. taking Spirulysat® concentrated to 100 mg of Phycocyanin and 15 mg of polysaccharides per day, allows an even greater reduction of undesirable side effects, such as peripheral neuropathy, induced by anti-cancer treatment, such as platinum salt treatments and in particular oxaliplatin-based treatments.

In particular, the inventors have shown that taking Spirulysat® concentrated at 100 mg of Phycocyanin and 15 mg of polysaccharides per day, allows to decrease from 45% to 10% the rate of patients undergoing oncological treatment with platinum salts and more particularly under oxaliplatin, presenting at least very serious undesirable side effects such as chemotherapy-induced peripheral neuropathy (see table 1 below). 90% of patients have only minor effects.

TABLE 1

| Spirulysat ® concentration | 0 mg/d | 100 mg/d |
|---|---|---|
| % patients with chemotherapy-induced neuropathy | 45% | 10% |

The effect of Spirulysat® is therefore dose-related. A minimum dose of 50 mg/day for an adult is recommended. For a child it is to be adapted to their body mass. The inventors have shown that a dose of 100 mg/day provides even better results, or a greater rate of decrease in side effects on the population.

The tolerance threshold of Spirulysat® from a toxicity point of view is beyond 1.4 litres of Spirulysat® per day for an adult, according to the latest studies on animal models. Some patients react much more easily to Spirulysat® and obtain good results with 5 ampoules per day (i.e. 50 mg/day of Phycocyanin and 7.5 mg/day of Spirulina polysaccharides). Others needed 10 ampoules per day (i.e. 100 mg/day of

Phycocyanin and 15 mg/day of Spirulina polysaccharides). A personalisation and an adaptation of the Spirulysat® intake is thus possible according to the patients, while remaining below the dosages described in the prior art.

Thus, Spirulysat®, combining several molecules of interest of Spirulina including Phycocyanin, and Spirulina polysaccharides, offers the opportunity of a customisation of concentration to meet the needs of patients.

In addition, taking Spirulysat® allows patients to tolerate their treatment better and thus, avoid or limit the dosage adjustment of the current treatment, contrary to what has been observed in 50% of cases of anti-cancer treatment without taking Spirulysat®. Indeed, the inventors have demonstrated that taking 20 mg/day of

Spirulysat® reduces to 10% the number of cases requiring a dosage adjustment (see table 2 below). With a dosage of 50 mg/day and 100 mg/day no further dosage adjustment is necessary.

Thus, patients better respect their treatment protocol, which contributes to reinforce its effectiveness, while benefiting from an improvement in their well-being in terms of: diet, sleep, physical activity, stress resistance.

TABLE 2

| Spirulysat ® concentration | 0 mg/d | 20 mg/d | 50 mg/d | 100 mg/d |
|---|---|---|---|---|
| % of cases of dosage adaptation | >50% | 10% | 0% | 0% |

Advantageously, Spirulysat® does not intervene as a medicinal treatment but as a therapeutic support contributing to making medical treatments more effective. This is a major innovation in terms of health: for the first time, the combination of a medical treatment and a natural food supplement results in a powerful and incredibly effective cocktail for the benefit of patients.

1. A composition for use in the treatment and/or prevention of chemically-induced peripheral neuropathy and its symptoms induced by an anti-cancer compound, said composition comprising an aqueous liquid extract, said aqueous liquid extract comprising Phycocyanin.

2. The composition for use according to claim 1, characterised in that said aqueous liquid extract further comprises Spirulina polysaccharides.

3. The composition for use according to claims 1, characterised in that said anti-cancer compound belongs to the group comprising a platinum salt, a taxane, an anthracycline.

4. The composition for use according to claim 3, characterised in that said anti-cancer compound is selected from platinum salts; preferably from the group consisting of cisplatin, carboplatin, oxaliplatin and mixtures thereof.

5. The composition for use according to claim 1 characterised in that said liquid aqueous extract comprises between 20 and 150 mg, preferably between 50 and 120 mg, very preferably 100 mg of Phycocyanin.

6. The composition for use according to claim 2, characterised in that said liquid aqueous extract further comprises between 5 and 25 mg, very preferably 20 mg, of Spirulina polysaccharides.

7. The composition for use according to claim 1, characterised in that said composition comprises only said liquid aqueous extract.

8. The composition for use according to claim 7, characterised in that it is administered in liquid form orally.

9. The composition for use according to claim 7, characterised in that said composition is administered at a dose between 20 mg/day and 150 mg/day, preferably between 50 mg/day and 150 mg/day, very preferably at 100 mg/day.

10. A use of an aqueous liquid extract, comprising Phycocyanin and optionally Spirulina polysaccharides, obtained from cyanobacteria for the treatment and/or prevention of chemotherapy-induced peripheral neuropathy and their symptoms induced by an anti-cancer compound.

11. The use of an aqueous liquid extract according to claim 10, characterised in that said anti-cancer compound belongs to the group comprising a platinum salt, a taxane, an anthracycline.

12. The use of an aqueous liquid extract according to claim 11, characterised in that said anti-cancer compound is selected from platinum salts; preferably from the group consisting of cisplatin, carboplatin, oxaliplatin and mixtures thereof.
